(19) United States
(12) Patent Application Publication
(10) Pub. No.: US 2007/0142788 A1
(43) Pub. Date: Jun. 21, 2007

(54) E-Module

(76) Inventors: Philippe Kohlbrenner, Kaltacker (CH); Markus Bingesser, Wuerenlos (CH); Johannes Felber, Dietikon (CH); Kurt Heinz, Buchs (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/609,521

(22) Filed: Dec. 12, 2006

(30) Foreign Application Priority Data
Dec. 13, 2005 (DE) 10 2005 059 494.8

(51) Int. Cl. A61M 5/00

(52) U.S. Cl. 604/207

(57) ABSTRACT

A dose setting module for an injection device including at least one dose switching device having associated therewith at least one conductive region and at least one non-conductive region, and at least one contacting device having associated therewith at least one conductive contact, and a method of detecting a dose setting in an injection device, whereby, to set a dose, the at least one dose switching device is turned to establish a connection between the at least one dose switching device and the at least one contact of the at least one contacting device, wherein when a connection has been established between at least a first conductive region of the at least one dose switching device and at least one contact of the at least one contacting device, a first signal is produced, and when a connection has been established between at least a second non-conductive region of the at least one dose switching device and at least one contact of the at least one contacting device, a second signal is produced, the dose setting is detected and/or evaluated using the signals.

### CROSS-REFERENCE TO RELATED APPLICATION

This application claims priority to German Application No. 10 2005 059 494.8, filed on Dec. 13, 2005, the content of which is incorporated herein by reference in its entirety.

### BACKGROUND

The present invention relates to devices for delivering, testing, administering, injecting or infusing substances, and to methods of making and using such devices. More particularly, it relates to medical devices for delivering, testing, administering, injecting or infusing selected amounts of substances, and to methods of making and such devices. More particularly, the present invention relates to an injection device comprising a dose metering module, and a method of detecting, determining or assessing a dose setting of the injection device. More particularly, the present invention relates to a dose metering module and a method of setting and dispensing a set dose of a dispensable product used in the medical field, whereby the actual quantity of the dispensable product can be determined by using electrically conductive contacts associated with the module and/or an injection device.

Generally speaking, the dose or amount of a dispensable product to be dispensed from an injection device is set or selected by rotating a dose setting knob and then dispensed by depressing the dose setting knob. Generally, known devices operate on the basis of mechanical principles and may be, therefore, complex to make, relatively inaccurate and susceptible to wear.

In administering or dispensing substance, particularly in the medical field, it is important to dispense an exact as possible a dose of the substance. Regarding injection devices, including those referred to as injection pens, for delivering insulin, hormone preparations, etc., it is valuable to be able to detect and/or monitor the process of setting a dose, and to emit a warning signal and/or prevent the dose from being dispensed if it is set incorrectly. Patent specification WO 93/16743 discloses a display unit for an injection device that is directed to as least some of these tasks.

### SUMMARY

An object of the present invention is to provide a device for setting, detecting and/or monitoring dose settings of injection devices which enables dose settings to be detected easily and accurately. Another object is to provide a method of setting, detecting and/or monitoring dose settings of injection devices which enables dose settings to be detected and monitored easily and accurately.

In one embodiment, the present invention comprises a dose setting module for an injection device comprising at least one dose switching device having associated therewith at least one conductive region and at least one non-conductive region, and at least one contacting device having associated therewith at least one conductive contact.

In one embodiment, the present invention comprises a method of detecting a dose setting of an injection device, wherein, when setting or selecting a dose, a dose switching device is turned establishing a first connective condition between the dose switching device and a contacting device, wherein when the first connective condition is established a first signal is produced, and when the dose switching device is turned to another position, another connective condition between the dose switching device and the contacting device is established and a second signal is produced, the dose setting detected using the signals.

In one embodiment, the present invention comprises a dose setting module for an injection device including at least one dose switching device with at least one conductive region and at least one non-conductive region, and having at least one contacting device with at least one conductive contact, and a method of detecting a dose setting in an injection device, whereby, to set a dose, the at least one dose switching device is turned in order to establish a connection between the at least one dose switching device and at least one contract of at least one contacting device, and when a connection has been established between at least a first conductive region of the at least one dose switching device and between at least one contact of the at least one contacting device, a first signal is output by the at least one contact, and when a connection has been established between at least a second non-conductive region of the at least one dose switching device and at least one contact of the at least one contacting device, a second signal is output by the at least one contact, and the dose setting is determined by evaluating the signals output by the at least one contact during the process of setting a dose.

In one embodiment, the device in accordance with the present invention, e.g., the dose setting module provided by the invention, comprises at least one dose switching device and at least one contacting device, and the device or dose setting module enables a dose of a dispensable or injectable product, e.g. a liquid such as insulin, hormone preparations, etc., set or selected by or on an injection device to be detected and monitored. In some preferred embodiments, the at least one dose switching device comprises a dose switching ring and the contacting device is used to make contact or establish a contact or connection with the at least one dose switching device. The at least one dose switching device comprises at least one conductive region, which is able to conduct electric current, and at least one non-conductive region, which can conduct no or virtually no electric current or at least has a higher ohmic resistance than the conductive region. The contacting device has at least one conductive region, in some embodiments comprising a conductive contact or slider which conducts electric current or which may have a low ohmic resistance.

In some embodiments, the dose switching device can be moved or turned into at last one first position in which at least one contact incorporating one of the conductive regions establishes an electrical connection or establishes an electrical contact. The dose switching device may also be moved or turned into at least one second position in which at least one of the contacts is in contact with or connected to at least one non-conductive region.

In some embodiments, the at least one dose switching device is maintained at a specific pre-defined electric potential, such as a positive potential for example, and the at least one contacting device is maintained at another pre-defined potential, such as a negative potential for example. The dose switching device may also be maintained at a negative potential and the contacting device may be maintained at a positive potential.

If, when a dose setting knob of an injection device is rotated to select or set a dose, and if a connection is established between a contact of the contacting device and a conductive region of the dose switching device, current is able to flow between the contact and the conductive region of the dose switching device, which can be measured at the contact, at the dose switching device or at another location. If a contact is connected to a non-conductive region, there is little or no flow of current between the dose switching device or the non-conductive region and the contact connected to it. The contacting device may also have two conductive contacts, which may be connected to one another via a voltage source. The dose setting module may be designed so that the contacts only establish a current circuit with the applied voltage if both contacts are electrically connected to or in contact with one of the conductive regions. If one or both contacts is or are in contact with or connected to a non-conductive region, no current flows through the current circuit formed by the contacts and voltage source because the current circuit is not closed but interrupted. In some embodiments, a check may be run or a measurement taken to establish whether a current is flowing between the contacts. When a current is flowing, it may be concluded that both contacts are in contact with a conductive region of the dose switching device. In some embodiments, the dose switching device can be turned or moved into at least one first position in which both contacts are in contact with a conductive region of the dose switching device, thereby enabling a current to flow. The dose switching device can also be turned to at least one second position in which at least one or both contacts is or are in contact with a non-conductive region of the dose switching device, in which case no current or only a low current flows through the interrupted circuit or to one of the two contacts.

In some embodiments, the at least one dose switching device has a cylindrical or annular shape, or may be provided in the form of a ring enclosing a threaded rod of the injection device. The at least one conductive region and the at least one non-conductive region may be disposed around the circumference of the dose switching device and, in some embodiments, may be provided in the form of mutually separate portions or sectors along the circumference of the dose switching device.

In some embodiments, the dose setting module may also have more than one or at least two dose switching devices, in which case the dose switching devices may be disposed along the longitudinal axis or along the longitudinal length of the dose setting module or injection device. The dose switching devices may adjoin one another or may be spaced at a distance apart from one another along the length of the injection device. All of the at least two dose switching devices are maintained at the same electric potential or are set to the same electric potential.

In some embodiments, the at least one conductive region of the at least one dose switching device is made from a conductive material or plastic, such as an inorganic or organic semiconductor. In some embodiments, the entire dose switching device may be made from a conductive material or a conductive plastic, in which case non-conductive or non-conducting regions are or may be provided, which may be created by a non-conductive plastic or provided in the form of cut-outs and thus formed by air.

In some embodiments, the at least one contacting device lies against the external face of the at least one dose switching device or moves or slides along it. In some embodiments, the at least one contacting device may be retained on the external face, in which case it is secured and lies permanently against the external face, or it may be spring-mounted and thus may lie flexibly on the external face of the dose switching device. If there are irregularities associated with the dose switching device, it follows the irregularities of the dose switching device.

In some preferred embodiments, the dose setting module may have two dose switching devices, in which case they may be provided along the longitudinal axis of the dose setting module and/or injection device and displaced relative to one another in the longitudinal direction. In some embodiments, it may be preferable for the dose setting module to have two contacting devices, in which case the contacting devices are mounted so that they can be displaced relative to one another in the longitudinal direction of the injection device. In some embodiments, a first dose switching device and a first contacting device lie opposite one another in the longitudinal direction of the dose setting module and a second dose switching device and a second contacting device lie opposite one another in the longitudinal direction of dose setting module. A dose of the dispensable product may be set by the dose setting module and, once the dose has been set, the contacts of the two contacting devices may be connected to the dose switching devices. If the contacting device and the dose switching device are set to a different potential from one another and if a contact is connected to a conductive region or portion of one of the two dose switching devices, current is able to flow to the contact. If a contact is connected to a non-conductive portion or region of one of the two dose switching devices, no current is able to flow to the contact. Consequently, by evaluating changes in the signal or output signal of the contacts, a conclusion may be drawn as to the status or setting of the dose setting module and/or an injection device with which it is associated.

In some embodiments, the first dose switching device may have two conductive regions and two non-conductive regions and the second dose switching device has one conductive region and one non-conductive region. The first contacting device may also specifically have two contacts and the second contacting device may have one contact, which does not overlap with either of the two contacts of the first contacting device in a circumferential direction and longitudinal direction of the dose switching device. In some preferred embodiments, three contacts are disposed so that they do not overlap in the circumferential direction of the injection device or dose setting module or do not lie opposite one another. The conductive regions and the non-conductive regions of the first dose switching device may also extend respectively across a quarter of the circumference of the first dose switching device, and the conductive region and non-conductive region of the second dose switching device may respectively extend across half of the circumference of the second dose switching device. As a result of this layout, up to eight settings of the dose setting module or injection device can be clearly allocated or defined.

If a contact is connected to or in contact with a conductive region of one of the dose switching devices, for example, a current is able to flow at this contact, a high signal can be output, or a logical “1” emitted. If a contact is connected to a non-conductive region of one of the dose switching devices or is in contact with it, it is preferable if no current is able to flow at this contact or only a weak current, , a low signal is emitted, or a logical “0” is emitted.

In some embodiments, the method of the present invention comprises detecting a dose setting of an injection device containing a dispensable or injectable product. For example, if a dose setting knob of an infusion or injection device is displaced or turned to set a dose, a dose switching device can be displaced or turned until a connection is established between the at least one dose switching device and at least one contact of at least one contacting device. If a connection is established between at least one conductive region of the at least one dose switching device and at least one contact of the at least one contacting device, for example, a first signal, a high signal or a logical “1” can be output by the at least one contact or detected on it. If a connection is established between at least one non-conductive region of the at least one dose switching device and at least one contact of the at least one contacting device, for example, a second signal different from the first signal, such as a low signal or a logical “0”, can be output by the at least one contact or can be detected at it. The term non-conductive region may also be construed as meaning a cut-out, which means that, in the at least one second position, there is no material connection between the at least one non-conductive region and the at least one contact, although this can be construed as a connection in the sense of an active connection.

In some embodiments, to set a dose of a dispensable product, two dose switching devices can be turned so that a connection is established between the two dose switching devices and two contacting devices. This being the case, a first connection can be established between a first dose switching device and two contacts of a first contacting device and a second connection can be established between a second dose switching device and one contact of a second contacting device.

In some embodiments, the first dose switching device and the first contacting device lie opposite one another along the longitudinal length of the dose setting module so that the two contacts of the contacting device lie against the external face of the first dose switching device or are actively connected to the dose switching device. If one or both of the two contacts of the first contacting device is or are actively connected to or in contact with a conductive region of the first dose switching device, a current is able to flow, causing a high signal to be emitted at one or both contacts. If one or both of the two contacts of the first contacting device is or are connected to or in contact with a non-conductive region of the first dose switching device, no current or only a low current is able to flow, causing a low signal to be emitted at one or both contacts.

In some embodiments, the second dose switching device and the second contacting device lie opposite one another along the longitudinal length of the dose setting module so that the contact of the second contacting device lies against the external face of the second dose switching device or is actively connected to the dose switching device. If the contact of the second contacting device is actively connected to or in contact with a conductive region of the second dose switching device, a current is able to flow, causing a high signal to be emitted at the contact. If the contact of the second contacting device is actively connected to or in contact with a non-conductive region of the second dose switching device, no current or only a low current is able to flow, causing a low signal to be output at the contact.

### BRIEF DESCRIPTION OF THE DRAWINGS

FIG. 1a illustrates a first embodiment of the present invention with a non-conductive plastic ring serving as a dose switching device, incorporating conductive regions made from conductive plastic and sliders serving as the contacting device lying permanently against the plastic ring;

FIG. 1b is a detail from another embodiment of the present invention with a non-conductive plastic ring serving as a dose switching device incorporating regions made from conductive plastic and spring-mounted sliders serving as the contacting device;

FIG. 2a is a cross-sectional view illustrating another embodiment of the present invention with a first and second dose switching device, and a first and second contacting device; and

FIG. 2b illustrates the embodiment of FIG. 2a in a plan view.

### DETAILED DESCRIPTION

With regard to fastening, mounting, attaching or connecting components of the present invention to form the invention, unless specifically described as otherwise, conventional mechanical fasteners and methods may be used. Other appropriate fastening or attachment methods include friction fitting, adhesives, welding, soldering, etc. The electrical system, electrical features or electrical components may comprise suitable electrical components, e.g., circuitry, wires, chips, boards, microprocessors, power sources, communication devices (e.g., transmitters, receivers, etc.), inputs, outputs, sensors, displays, control components, etc. Generally, unless otherwise indicated, the materials for making the device of the present invention and/or its components may be selected from appropriate materials such as metals, metallic alloys, ceramics, plastics, etc.

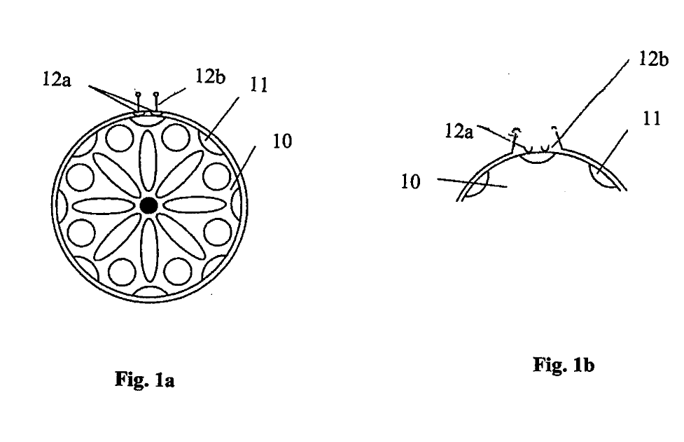

FIG. 1a illustrates one embodiment of the present invention, in which a dose setting module for incorporation into an injection device is provided in the form of a dose switching device comprising a plastic ring 10 made from a non-conductive plastic on or in which conductive regions 11 of conductive plastic or metal are formed, which plastic ring 10 is resiliently pressed against or spring-biased onto the contacts or held in the radial direction against the contacts or remains connected to the contacts in the radial direction. The plastic ring 10 is connected to a dose-setting rotating wheel or to a rotating knob of an injection device or is fitted on or in the latter. The dose setting module also has two contacts or sliders 12a, 12b serving as a contacting device, which are retained or mounted in the radial direction against the external face of the plastic ring 10. When the dose setting knob or dose setting wheel of the injection device is rotated or displaced, the plastic ring 10 is able to rotate relative to the contacts 12a, 12b so that different regions of the plastic ring 10 are able to move into contact with or actively connect to the contacts or sliders 12a, 12b. The sliders and the conductive regions 11 of the dose setting device or plastic ring 10 may be set to a different electric potential or have a different electric potential.

A voltage may also be applied to the terminals of the contacts 12a, 12b. If both contacts 12a, 12b are connected to a conductive region 11 of the dose switching device 10, a current is able to flow from the one contact 12a, 12b to the other contact 12a, 12b via the conductive region 11, thereby establishing a circuit. If at least one or if both of the contacts 12a, 12b is or are in contact with or connected to a non-conductive region, no current is able to flow, thereby enabling a conclusion to be drawn as to what setting the dose setting module is currently in and to what region of the dose switching device the contacts are connected. In FIG. 1, the contacts 12a, 12b and a conductive region 11 of the dose switching device are in contact with one another, which means that a current is able to flow between the sliders and the conductive region 11. This current may be recorded or measured at the sliders, for example. If the dose setting knob were rotated further, clockwise or anti-clockwise, by 22.5°, for example, at least one of the contacts 12a, 12b would move into contact with a non-conductive region of the plastic ring 10, in which case no current or only a low current would be able to flow.

For example, both contacts 12a, 12b to which a voltage is applied may be connected to a conductive region 11 of the dose switching device or the plastic ring 10 so that the two contacts 12a, 12b are connected to one another by a current circuit passing across the conductive region 11. The conductive region 11 has a low or no ohmic resistance, thereby resulting in a current which can be detected or measured, thus making it possible to determine that a conductive region 11 is lying against the two contacts 12a, 12b. In particular, several or all of the conductive regions 11 of the dose switching device or the plastic ring 10 may have a different ohmic resistance, thereby resulting in a current of varying intensity, depending on which conductive region 11 is lying against the contacts 12a, 12b. The current may be measured, thereby making it possible to determine which conductive region is lying against the contacts 12a, 12b on the basis of the measured current or the intensity of the measured electric current. In another position or dose setting, a conductive region 11 may lie against the left-hand contact 12a and a non-conductive region against the right-hand contact 12b. The non-conductive region lying against the right-hand contact 12b has a high ohmic resistance so that no circuit is established between the contacts 12a, 12b, thereby resulting in no current or only a low current. The same situation occurs if the left-hand contact 12a is lying against a non-conductive region and the right-hand contact 12b is lying against a conductive region 11 of the plastic ring 10. In another position or dose setting, both contacts 12a, 12b are connected to non-conductive regions of the dose switching device or plastic ring 10, in which case no circuit is established between the contacts and the non-conducive regions due to the high ohmic properties or resistance properties of the non-conductive region, thereby resulting in no current flow or only a low current flow.

FIG. 1b illustrates the dose setting module from FIG. 1a in which the sliders or contacts 12a, 12b are spring-mounted or resiliently disposed so that they lie against the external face of the plastic ring 10. The contacts are able to move radially relative to the plastic ring 10 yet are pressed or retained against the plastic ring 10.

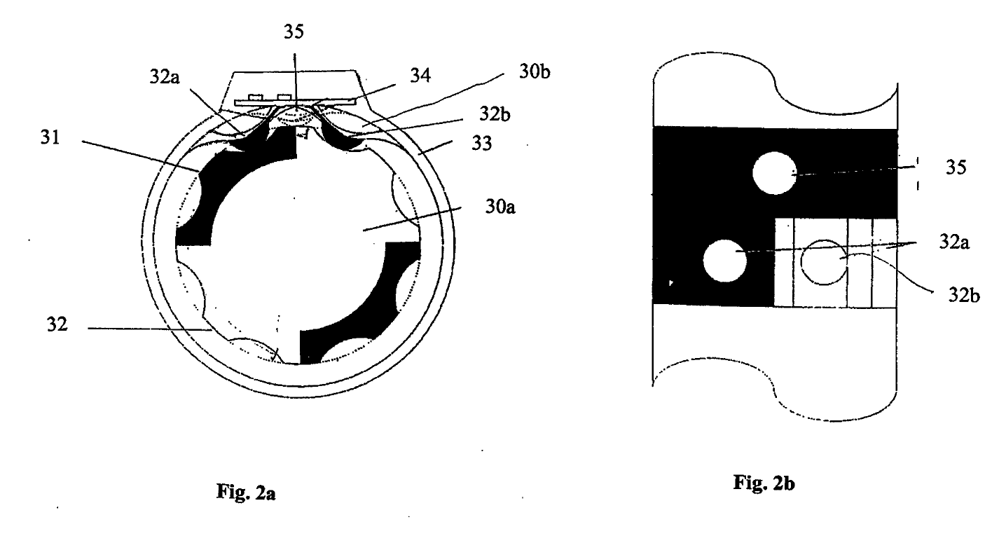

FIGS. 2a (sectional view) and 2b (plan view) illustrate another embodiment of the dose setting module of the present invention. The dose setting module has two dose switching devices 30a, 30b disposed one after the other or adjacent to one another in the longitudinal direction or along the longitudinal length of the dose setting module. The dose setting module also has two contacting devices disposed one after the other along the longitudinal length of the dose setting module. The first dose switching device 30a has two conductive regions 31 and two non-conductive regions 32 alternating with one another along the circumference of the dose switching device 30a. The first dose switching device 30a has notches, or indentations, cut-outs or relieved areas, in which contacts 32a, 32b are able to locate or by which the contacts 32a, 32b can be actively connected to the dose switching device 30a. Disposed in the same position along the length of the dose setting module is a first contacting device with two contacts 32a, 32b, which are mounted so that they touch the external face of the dose switching device 30a. The dose setting module also has a second dose switching device 30b with a conductive region and a non-conductive region. The second dose switching device 30b is disposed opposite a second contacting device with one contact 35, which is disposed in the circumferential direction of the dose setting module between the two contacts 32a, 32b of the first contacting device 30a, or is disposed so that the contact 35 of the second contacting device does not overlap with the two contacts 32a, 32b of the first contacting device 30a in the circumferential direction.

FIG. 2b shows the device from FIG. 2a in a plan view. On the dose switching devices 30a, 30b or on the conductive regions of the dose switching device is a first positive or negative potential, i.e., the switching devices or the conductive regions are at a first potential. The contacts 32a, 32b, 35 are set to a second potential, in some embodiments, preferably different from the first potential.

In the position illustrated in FIGS. 2a and 2b, a first contact 32b of the first contacting device is connected or operably coupled to a non-conductive region 32 of the first dose switching device 30a so that there is no current flow or only a low current flow, resulting in the output of a logical “0” or a low signal at the contact 32b. A second contact 32a of the first contacting device is connected or operably coupled to a conductive region 31 of the first dose switching device so that a current flows, causing the output of a logical “1” or a high signal at the contact 32. A contact 35 of the second contacting device is also connected or operably coupled to a conductive region 31 of the second dose switching device 30b causing a current flow and hence the output of a logical “1” or a high signal at the contact 35. If the dose switching device 30a, 30b is turned in the clockwise direction, e.g., by one position, by turning or moving the rotating knob or dose setting knob of the injection device for example, both contacts 32a, 32b lie on or are operably coupled to a conductive region 31 of the first dose switching device 30a so that a current flows at both contacts or a high signal is output. The contact 35 of the second dose switching device 30b also lies on the conductive region of the second dose switching device 30b, causing a current to flow at all three contacts or a logical “1” to be output. Using this embodiment of the present invention, i.e., the embodiment illustrated in FIGS. 2a and 2b, eight dose settings can be set, each setting or position being clearly defined by the signals occurring at the contacts. Up to eight different dose settings can be clearly allocated or detected with two dose switching devices 30a, 30b using the design illustrated in FIGS. 2a and 2b.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A dose setting module for an injection device, the module comprising at least one dose switching device having associated therewith at least one conductive region and at least one non-conductive region, and at least one contacting device having associated therewith at least one conductive contact, wherein one of the dose switching device and contacting device is moveable relative to the other.

2. A dose setting module for an injection device, the module comprising:
at least one dose switching device with at least one conductive region and at least one region which is less conductive or non-conductive compared to the conductive region, and
at least one contacting device with at least one conductive contact, wherein
the dose switching device and the contacting device can be moved relative to one another into at least one first position in which the at least one contact is electrically connected to the at least one conductive region and into at least one second position in which the at least one contact is not electrically connected to the at least one conductive region, and a first signal can be detected or measured in the first position and a second signal different from the first signal can be detected or measured in the second position.

3. The dose setting module as claimed in claim 2, wherein the contacting device has two conductive contacts, and both contacts are electrically connected to the at least one conductive region in the at least one first position, and at least one contact is not in contact with the at least one conductive region in the at least one second position, and a current flowing through the circuit formed by the two contacts and the conductive region in the first position can be measured and no current flow can be measured in the second position.

4. The dose setting module as claimed in claim 2, wherein the at least one dose switching device is one of annular or cylindrical and the at least one conductive region and the at least one region with a lesser or no conductivity comprises portions of the circumference of the dose switching device.

5. The dose setting module as claimed in claim 2, wherein the dose setting module comprises at least two dose switching devices, and the dose switching devices are disposed generally along the length of the injection device.

6. The dose setting module as claimed in claim 5, wherein the at least one conductive region is made from one of a conductive plastic, an inorganic semiconductor or an organic semiconductor.

7. The dose setting module as claimed in claim 5, wherein the at least one region with a lesser or no conductivity is made from one of a plastic with low or no conductivity or is an empty space defined by the dose switching device.

8. The dose setting module as claimed in claim 5, wherein the at least one dose switching device is made from one of a conductive plastic, an inorganic semiconductor or an organic semiconductor.

9. The dose setting module as claimed in claim 5, wherein the at least one contacting device is designed so that the at least one contact lies permanently against the external face of one of the at least one contacting device.

10. The dose setting module as claimed in claim 5, wherein the at least one contacting device is designed so that the at least one contact is resiliently mounted on the external face of one of the at least one contacting devices.

11. The dose setting module as claimed in claim 2, wherein the dose setting module comprises two dose switching devices and two contacting devices, and the dose switching devices and the contacting devices are disposed generally along the longitudinal axis of the dose setting module, and a first dose switching device and a first contacting device lie opposite one another in the longitudinal direction of the dose setting module, and a second dose switching device and a second contacting device lie opposite one another in the longitudinal direction of the dose setting module.

12. The dose setting module as claimed in claim 11, wherein the first dose switching device has two conductive regions and two less conductive or non-conductive regions and the second dose switching device has one conductive region and one slightly conductive or non-conductive region, and the first contacting device has two contacts and the second contacting device has one contact which does not overlap with either of the two contacts of the first contacting device in the circumferential direction and longitudinal direction of the dose switching device.

13. The dose setting module as claimed in claim 12, wherein the conductive regions and slightly conductive or non-conductive regions of the first dose switching device respectively extend across a quarter of the circumference of the first dose switching device and the conductive region and the slightly conductive or non-conductive region of the second dose switching device respectively extend across half the circumference of the second dose switching device.

14. A method of detecting a dose setting of an injection device, wherein, when setting or selecting a dose, a dose switching device is turned establishing a first connective condition between the dose switching device and a contacting device, wherein when the first connective condition is established a first signal is produced, and when the dose switching device is turned to another position, another connective condition between the dose switching device and the contacting device is established and a second signal is produced, the dose setting detected using the signals.

15. A method of detecting a dose setting in an injection device, whereby, to set a dose of a dispensable product, at least one dose switching device with at least one conductive region and at least one slightly conductive or non-conductive region and at least one contacting device with at least one conductive contact are turned relative to one another into one of at least one first position and at least one second position, and at least one contact is electrically connected to the at least one conductive region and a first signal, in particular a current or a voltage, can be detected between the at least one contact and the conductive region in the at least one first position, and, in the at least one second position, at least one contact is not electrically connected to a conductive region and a second signal different from the first signal can be detected.

16. The method as claimed in claim 15, wherein, to set a dose of a dispensable product, two dose switching devices and two contacting devices are turned relative to one another into one of at least one first position and at least one second position, and the two contacts of the first contacting device are electrically connected to the at least one conductive region of the first dose switching device and the contact of the second contacting device is electrically connected to the at least one conductive region of the second dose switching device in the at least one first position, and, in the at least one second position, at least one contact of the first contacting device is not electrically connected to the at least one conductive region of the first dose switching device and/or the contact of the second contacting device is not electrically connected to the at least one conductive region of the second dose switching device.

17. The method as claimed in claim 16, wherein the first signal is a high signal and the second signal is a low signal.
